(19) United States
(12) Patent Application Publication
(10) Pub. No.: US 2020/0206011 A1
(43) Pub. Date: Jul. 2, 2020

(54) SPLINT AND OPERATION METHOD THEREOF

(71) Applicant: BOE TECHNOLOGY GROUP CO., LTD., Beijing (CN)

(72) Inventors: Zhihong DU, Beijing (CN); Wenbo Li, Beijing (CN)

(21) Appl. No.: 16/534,182

(22) Filed: Aug. 7, 2019

(30) Foreign Application Priority Data
Jan. 2, 2019 (CN) 201910002868.8

(51) Int. Cl. A61F 5/058

(52) U.S. Cl. CPC A61F 5/05841; A61F 13/04

(57) ABSTRACT

A splint and an operation method thereof are provided. The splint includes a plurality of plates and a splint fastening device. The splint fastening device is connected to the plurality of plates and configured to allow the plurality of plates to be relatively close to each other or relatively away from each other. At least one plate of the plurality of plates includes two sub-plates that are relatively rotatable, and the splint is capable of being bent or restored by relative rotation of the two sub-plates.

### CROSS-REFERENCE

The present application claims priority of Chinese patent application No. 201910002868.8, filed on Jan. 2, 2019, the disclosure of which is incorporated herein by reference as part of the application.

### TECHNICAL FIELD

Embodiments of the present disclosure relate to a splint and an operation method thereof.

### BACKGROUND

In medical fields, plasters are commonly used to fix injured limbs of a patient to protect the injured limbs and promote wound healing. For example, a plaster bandage and a plaster cast can be used to fix the injured limb of the patient. With the development of technologies, polymer bandages and splints are widely used in medical fields, and become a mainstream fixing method because of the advantages such as high strength, light weight, thin thickness, breathability, waterproofness, non-skin-allergy, ability to transmit X-rays, easy disassembly, and so on.

### SUMMARY

At least one embodiment of the present disclosure provides a splint, which comprises: a plurality of plates and a splint fastening device connected to the plurality of plates and configured to allow the plurality of plates to be relatively close to each other or relatively away from each other. At least one plate of the plurality of plates comprises two sub-plates that are relatively rotatable, and the splint is capable of being bent or restored by relative rotation of the two sub-plates.

For example, in the splint provided by an embodiment of the present disclosure, the at least one plate comprises a first plate, and the first plate comprises a first sub-plate, a second sub-plate, and a first rotating joint, the first sub-plate and the second sub-plate are both connected to the first rotating joint, and the first sub-plate and the second sub-plate are in surface contact with each other, and are relatively rotatable in a plane in which a contact surface between the first sub-plate and the second sub-plate is located.

For example, in the splint provided by an embodiment of the present disclosure, the first rotating joint comprises a fixing unit and a rotating unit, the fixing unit is connected to one of the first sub-plate and the second sub-plate, and the rotating unit is connected to the fixing unit and another one of the first sub-plate and the second sub-plate.

For example, in the splint provided by an embodiment of the present disclosure, the fixing unit comprises a fixing shaft, and the fixing shaft is fixed on one of the first sub-plate and the second sub-plate, the rotating unit comprises a bearing, an inner ring of the bearing is fixedly connected to the fixing shaft, and an outer ring of the bearing is fixedly connected to another one of the first sub-plate and the second sub-plate.

For example, in the splint provided by an embodiment of the present disclosure, a portion, which is connected to the first rotating joint, of the first sub-plate comprises a first connecting portion and a second connecting portion that are stacked, a hollow structure is between the first connecting portion and the second connecting portion, and a portion, which is connected to the first rotating joint, of the second sub-plate is in the hollow structure.

For example, the splint provided by an embodiment of the present disclosure further comprises at least one positioning component, and the positioning component is on the first sub-plate.

For example, in the splint provided by an embodiment of the present disclosure, the positioning component comprises a pin and a pin hole, the pin hole extends through the first connecting portion and the second connecting portion, and the pin is in the pin hole and is linearly movable in the pin hole; and a length of the pin is greater than a sum of a thickness of the first connecting portion and a thickness of the hollow structure, or greater than a sum of a thickness of the second connecting portion and a thickness of the hollow structure.

For example, in the splint provided by an embodiment of the present disclosure, the positioning component further comprises a hollow bolt and a spring, and the pin comprises a convex plate, the hollow bolt is on one end of the pin hole, and the pin is in the hollow bolt, one end of the spring is connected to the hollow bolt, and another end of the spring is connected to the convex plate.

For example, in the splint provided by an embodiment of the present disclosure, the portion, which is connected to the first rotating joint, of the second sub-plate comprises a plurality of through holes, and the plurality of through holes are configured to allow the first sub-plate and the second sub-plate to be relatively fixed and respectively have a plurality of different bending angles in a case where the pin is respectively inserted into the plurality of through holes.

For example, in the splint provided by an embodiment of the present disclosure, the at least one plate comprises a second plate, the second plate comprises a third sub-plate, a fourth sub-plate, and a second rotating joint, and the third sub-plate and the fourth sub-plate are both connected to the second rotating joint, and are configured to adopt the second rotating joint as a rotating axis to rotate with respect to the second rotating joint.

For example, in the splint provided by an embodiment of the present disclosure, the second rotating joint comprises a hinge structure.

For example, in the splint provided by an embodiment of the present disclosure, the at least one plate comprises a plurality of first plates and a plurality of second plates, each of the plurality of first plates comprises a first sub-plate, a second sub-plate, and a first rotating joint, the first sub-plate and the second sub-plate are both connected to the first rotating joint, and the first sub-plate and the second sub-plate are in surface contact with each other and are relatively rotatable in a plane in which a contact surface between the first sub-plate and the second sub-plate is located, each of the plurality of second plates comprises a third sub-plate, a fourth sub-plate, and a second rotating joint, and the third sub-plate and the fourth sub-plate are both connected to the second rotating joint, and are configured to adopt the second rotating joint as a rotating axis to rotate with respect to the second rotating joint, the plurality of first plates are oppositely disposed, the plurality of second plates are oppositely disposed, and the first plates are adjacent to the second plates.

For example, in the splint provided by an embodiment of the present disclosure, the splint fastening device comprises: at least one drawstring movably connected to the plurality of plates, and at least one fastening drive device on one or more of the plurality of plates, connected to the at least one drawstring and configured to be controllable to drive the at least one drawstring, so that the drawstring is capable of being tightened or relaxed under driving function of the fastening drive device to drive the plurality of plates to be relatively close to each other or relatively away from each other.

For example, in the splint provided by an embodiment of the present disclosure, the at least one drawstring comprises a plurality of drawstrings, and the at least one fastening drive device comprises a plurality of fastening drive devices, the plurality of drawstrings are connected to the plurality of fastening drive devices in one-to-one correspondence, and the plurality of drawstrings are movably connected to plates of different portions of the splint that are capable of being bent, respectively.

For example, the splint provided by an embodiment of the present disclosure further comprises a pulling force detection circuit, and the pulling force detection circuit is coupled to the drawstring and is configured to detect a pulling force applied to the drawstring.

For example, the splint provided by an embodiment of the present disclosure further comprises a display component, and the display component is in signal connection with the pulling force detection circuit, and is configured to display a pulling force detection result of the pulling force detection circuit.

For example, in the splint provided by an embodiment of the present disclosure, the portion, which is connected to the first rotating joint, of the second sub-plate comprises a plurality of through holes, and the plurality of through holes are configured to allow the first sub-plate and the second sub-plate to be relatively fixed and respectively have a plurality of different bending angles in a case where the pin is respectively inserted into the plurality of through holes; the at least one plate comprises a second plate, the second plate comprises a third sub-plate, a fourth sub-plate, and a second rotating joint, and the third sub-plate and the fourth sub-plate are both connected to the second rotating joint, and are configured to adopt the second rotating joint as a rotating axis to rotate with respect to the second rotating joint, and the second rotating joint comprises a hinge structure; the at least one plate comprises a plurality of first plates and a plurality of second plates, the plurality of first plates are oppositely disposed, the plurality of second plates are oppositely disposed, and the first plates are adjacent to the second plates; the splint fastening device comprises: at least one drawstring movably connected to the plurality of plates; and at least one fastening drive device on one or more of the plurality of plates, connected to the at least one drawstring and configured to be controllable to drive the at least one drawstring, so that the drawstring is capable of being tightened or relaxed under driving function of the fastening drive device to drive the plurality of plates to be relatively close to each other or relatively away from each other; the at least one drawstring comprises a plurality of drawstrings, and the at least one fastening drive device comprises a plurality of fastening drive devices, the plurality of drawstrings are connected to the plurality of fastening drive devices in one-to-one correspondence, and the plurality of drawstrings are movably connected to plates of different portions of the splint that are capable of being bent, respectively; the splint further comprises a pulling force detection circuit, and the pulling force detection circuit is coupled to the drawstring and is configured to detect a pulling force applied to the drawstring; and the splint further comprises a display component, and the display component is in signal connection with the pulling force detection circuit, and is configured to display a pulling force detection result of the pulling force detection circuit.

For example, in the splint provided by an embodiment of the present disclosure, the portion, which is connected to the first rotating joint, of the second sub-plate comprises a plurality of through holes, and the plurality of through holes are configured to allow the first sub-plate and the second sub-plate to be relatively fixed and respectively have a plurality of different bending angles in a case where the pin is respectively inserted into the plurality of through holes; the splint fastening device comprises: at least one drawstring movably connected to the plurality of plates; and at least one fastening drive device on one or more of the plurality of plates, connected to the at least one drawstring and configured to be controllable to drive the at least one drawstring, so that the drawstring is capable of being tightened or relaxed under driving function of the fastening drive device to drive the plurality of plates to be relatively close to each other or relatively away from each other; the at least one drawstring comprises a plurality of drawstrings, and the at least one fastening drive device comprises a plurality of fastening drive devices, the plurality of drawstrings are connected to the plurality of fastening drive devices in one-to-one correspondence, and the plurality of drawstrings are movably connected to plates of different portions of the splint that are capable of being bent, respectively; the splint further comprises a pulling force detection circuit, and the pulling force detection circuit is coupled to the drawstring and is configured to detect a pulling force applied to the drawstring; and the splint further comprises a display component, and the display component is in signal connection with the pulling force detection circuit, and is configured to display a pulling force detection result of the pulling force detection circuit.

At least one embodiment of the present disclosure further provides an operation method of the splint provided by any one of the embodiments of the present disclosure, which comprises: allowing a using object to get into a space surrounded by the plurality of plates; allowing the splint to be bent and fixed by a positioning component; and using the splint fastening device to allow the plurality of plates to be relatively close to each other and to be attached to the using object.

At least one embodiment of the present disclosure further provides an operation method of the splint provided by any one of the embodiments of the present disclosure, which comprises: using the splint fastening device to allow the plurality of plates to be relatively away from each other and away from a using object; adjusting a positioning component and restoring the splint; and allowing the using object to move out of a space surrounded by the plurality of plates.

### BRIEF DESCRIPTION OF THE DRAWINGS

In order to demonstrate clearly technical solutions of the embodiments of the present disclosure, the accompanying drawings in relevant embodiments of the present disclosure will be introduced briefly. It is apparent that the drawings may only relate to some embodiments of the disclosure and not intended to limit the present disclosure.

FIG. 1 is a stereoscopic schematic diagram of a splint provided by some embodiments of the present disclosure;

FIG. 2 is a sectional schematic diagram of a first rotating joint of the splint illustrated in FIG. 1;

FIG. 3 is a partial enlarged diagram of a second rotating joint of the splint illustrated in FIG. 1;

FIG. 4 is a stereoscopic schematic diagram of another splint provided by some embodiments of the present disclosure;

FIG. 5 is a flow diagram of an operation method of a splint provided by some embodiments of the present disclosure; and

FIG. 6 is a flow diagram of another operation method of a splint provided by some embodiments of the present disclosure.

### DETAILED DESCRIPTION

In order to make objects, technical details and advantages of the embodiments of the disclosure apparent, the technical solutions of the embodiment will be described in a clearly and fully understandable way in connection with the drawings related to the embodiments of the disclosure. It is apparent that the described embodiments are just a part but not all of the embodiments of the disclosure. Based on the described embodiments herein, those skilled in the art may obtain other embodiment, without any creative work, which shall be within the scope of the disclosure.

Unless otherwise defined, all the technical and scientific terms used herein have the same meanings as commonly understood by one of ordinary skill in the art to which the present disclosure belongs. The terms, such as “first,” “second,” or the like, which are used in the description and the claims of the present disclosure, are not intended to indicate any sequence, amount or importance, but for distinguishing various components. The terms, such as “comprise/comprising,” “comprise/comprising,” or the like are intended to specify that the elements or the objects stated before these terms encompass the elements or the objects and equivalents thereof listed after these terms, but not preclude other elements or objects. The terms, such as “connect/connecting/connected,” “couple/coupling/coupled” or the like, are not limited to a physical connection or mechanical connection, but may comprise an electrical connection/coupling, directly or indirectly. The terms, “on,” “under,” “left,” “right,” or the like are only used to indicate relative position relationship, and when the position of the object which is described is changed, the relative position relationship may be changed accordingly.

In a case where a polymer bandage and a splint are used for fixing, it is required to wrap a plurality of splints separated from each other onto the injured limb of the patient by the polymer bandage. Therefore, it is difficult and spends long time to wrap the splints. Moreover, after the splints are fixed, problems such as looseness and displacement are easily to occur, which not only affect the fixing effect of the splints, but also may cause secondary injury to the patient and aggravate the patient's condition. Furthermore, the frequent disassembly of the splints increases the workload of the medical staff and reduces the work efficiency.

At least one embodiment of the present disclosure provides a splint and an operation method thereof, the splint can adjust a bending angle and be fixed, and the splint has a wide application and can enable the user to adjust quickly and easily, thereby reducing the workload of the user, improving the work efficiency, and improving the user's experience. The splint provided by at least some of the embodiments can also monitor the tightness degree.

In the following, the embodiments of the present disclosure are described in detail with reference to the accompanying drawings. It should be noted that the same reference numerals used in different drawings refer to the same components that are described.

At least one embodiment of the present disclosure provides a splint, and the splint comprises a plurality of plates and a splint fastening device. The splint fastening device is connected to the plurality of plates and is configured to allow the plurality of plates to be relatively close to each other or relatively away from each other. At least one plate of the plurality of plates comprises two sub-plates that are relatively rotatable, and the splint is capable of being bent or restored by relative rotation of the two sub-plates.

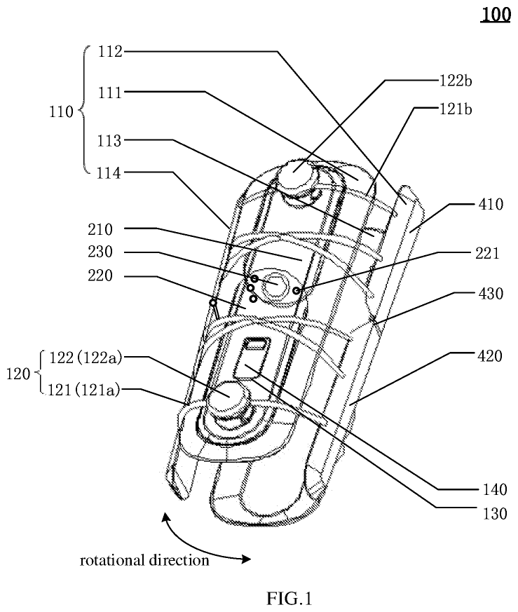

FIG. 1 is a stereoscopic schematic diagram of a splint provided by some embodiments of the present disclosure. As illustrated in FIG. 1, a splint 100 comprises a plurality of plates 110 and a splint fastening device 120.

The plurality of plates 110 of the splint 100 comprise first plates 111 and 113, and second plates 112 and 114. For example, the first plate 111 and the first plate 113 are oppositely disposed, and the second plate 112 and the second plate 114 are oppositely disposed. A space surrounded by the plurality of plates 110 can accommodate a user's limb, for example, a patient's injured limb, such as a leg, an arm, or the like, so as to fix and protect the injured limb. For example, each of the plates can be made of a polymer material, such as a glass fiber, a polyurethane, a resin, or the like, and other suitable materials may also be used. The embodiments of the present disclosure are not limited in this aspect.

It should be noted that, in the embodiments of the present disclosure, the number of the plurality of plates 110 is not limited to four in the above case, and may also be other suitable values, such as 2-6, and the embodiments of the present disclosure are not limited thereto. Sizes of the plurality of plates 110 are not limited, and can be determined according to the size of the user's limb or empirical values. Sizes of the plurality of plates 110 can be the same or different. For example, in an example, sizes of the first plate 111 and the first plate 113 are the same, sizes of the second plate 112 and the second plate 114 are the same, and the size of the first plate 111 and the first plate 113 is greater than the size of the second plate 112 and the second plate 114. In this way, the plurality of plates 110 can better fit the user's limb, thereby providing better functions of fixing, protection, and so on.

For example, the splint fastening device 120 is connected to the plurality of plates 110 and configured to allow the plurality of plates 110 to be relatively close to each other or relatively away from each other. The splint fastening device 120 comprises at least one drawstring 121 and at least one fastening drive device 122. For example, in some examples, the at least one drawstring 121 comprises a first drawstring 121a and a second drawstring 121b, and the at least one fastening drive device 122 comprises a first fastening drive device 122a and a second fastening drive device 122b.

The first drawstring 121a and the second drawstring 121b are movably connected to the plurality of plates 110. For example, a plurality of through holes are provided at edges of the first plates 111 and 113 and the second plates 112 and 114, and the first drawstring 121a and the second drawstring 121b pass through the through holes of each plate, and thus are movably connected to the plurality of plates 110. For example, the first drawstring 121a and the second drawstring 121b respectively pass through the plurality of through holes in an S-shaped cross manner or other suitable manners, different manners may have different effects on lengths of the first drawstring 121a and the second drawstring 121b and the fastening degree of the connection between the plurality of plates 110, and the manners may be determined according to actual needs.

The first fastening drive device 122a and the second fastening drive device 122b are disposed on one or more of the plurality of plates 110, for example, disposed on the first plate 111. The first fastening drive device 122a and the second fastening drive device 122b are respectively connected to the first drawstring 121a and the second drawstring 121b in one-to-one correspondence, and are configured to be controllable to respectively drive the first drawstring 121a and the second drawstring 121b, so that the first drawstring 121a and the second drawstring 121b can be tightened or relaxed under driving function of the first fastening drive device 122a and the second fastening drive device 122b, respectively, thereby driving the plurality of plates 110 to be relatively close to each other or relatively away from each other.

In a case where the first drawstring 121a and the second drawstring 121b are relaxed to allow the plurality of plates 110 to be relatively away from each other, the space surrounded by the plurality of plates 110 becomes larger; and in a case where the first drawstring 121a and the second drawstring 121b are tightened to allow the plurality of plates 110 to be relatively close to each other, the space surrounded by the plurality of plates 110 becomes smaller. Therefore, by tightening or relaxing the first drawstring 121a and the second drawstring 121b using the first fastening drive device 122a and the second fastening drive device 122b, the space surrounded by the plurality of plates 110 can be larger or smaller according to user's requirements. In a case where the splint 100 is used for different users and for different limbs, the splint 100 can be attached to the limb well, thereby realizing the functions of fixing, protection, and so on.

It should be noted that, in the embodiments of the present disclosure, the number of the drawstrings 121 and the number of the fastening drive devices 122 are not limited, and may be one, two or other values, which may be determined according to actual needs. For example, the number of the drawstrings 121 is equal to the number of the fastening drive devices 122, so as to achieve a one-to-one corresponding connection of the drawstrings 121 and the fastening drive devices 122.

It should be noted that, in the embodiments of the present disclosure, the specific implementation manner of the fastening drive device 122 is not limited. For example, in some embodiments, the fastening drive device 122 can be implemented in a form of a combination of a knob, a drawstring reel slot, and a fixed shaft, thereby reducing the cost. For example, in other embodiments, the fastening drive device 122 can be implemented in a form of a combination of a motor, a motor mount, and a drawstring reel slot, thereby improving adjustment efficiency and saving time and effort.

For example, at least one of the plurality of plates 110 comprises two sub-plates that are relatively rotatable, and the splint 100 can be bent or restored by relative rotation of the two sub-plates. For example, in some examples, as illustrated in FIG. 1, each plate of the plurality of plates 110 comprises two sub-plates that are relatively rotatable.

For example, the first plate 111 comprises a first sub-plate 210, a second sub-plate 220, and a first rotating joint 230. The first sub-plate 210 and the second sub-plate 220 are both connected to the first rotating joint 230, and the first sub-plate 210 and the second sub-plate 220 are in surface contact with each other, and are relatively rotatable in a plane in which a contact surface between the first sub-plate 210 and the second sub-plate 220 are located. Similarly, the first plate 113 disposed opposite to the first plate 111 also has a similar or identical structure.

For example, the second plate 112 comprises a third sub-plate 410, a fourth sub-plate 420, and a second rotating joint 430. The third sub-plate 410 and the fourth sub-plate 420 are both connected to the second rotating joint 430, and are configured to adopt the second rotating joint 430 as a rotating axis to rotate with respect to the second rotating joint 430. Similarly, the second plate 114 disposed opposite to the second plate 112 also has a similar or identical structure.

Therefore, the splint 100 can be bent or restored as a whole in the rotational direction illustrated in FIG. 1. In a case where the user uses the splint 100, the user's limb can get into the space surrounded by the plurality of plates 110 from an end of the splint 100, and the bending angle of the splint 100 can be adjusted, so that the splint 100 is bent and the bending angle is substantially the same as the bending angle of the user's limb. Then, the splint 100 is fixed by a positioning component to maintain the bending angle. By adjusting the fastening drive device 122, the drawstring 121 is tightened, so that the plurality of plates 110 are attached to the limb, thereby realizing the functions of fixing, protection, and so on. Of course, the embodiments of the present disclosure are not limited thereto, the splint 100 may not be bent, and the linear state illustrated in FIG. 1 is maintained during use, which may be determined according to the shape of the limb of the user. The positioning component is described in detail later, and is not described here.

In this way, the splint 100 is not only suitable for a linear limb, such as a crus, a forearm, etc., but also suitable for a polygonal limb, such as an elbow, a knee, etc., and has a wide application. The splint 100 can be adjusted quickly and easily by the user, which reduces the workload of the user, improves the work efficiency, and improves the user's experience. Moreover, the splint 100 is formed to an integrated structure rather than a plurality of separate components. Compared to the conventional separate splints, the splint 100 is easier to be stored when not in use, and it is not easy to lose its components.

In the splint 100, the plurality of drawstrings 121 (for example, the first drawstring 121a and the second drawstring 121b) are movably connected to plates of different portions of the splint 100 that can be bent, respectively, so that a portion of the plate that is connected to the first drawstring 121a (such as the second sub-plate 220 and the fourth sub-plate 420) and a portion of the plate that is connected to the second drawstring 121b (such as the first sub-plate 210 and the third sub-plate 410) can be respectively and independently adjusted with different tightness degrees. In a case where the splint 100 is used for a polygonal limb, for example, for an elbow, a knee, or the like, because sizes of the upper arm and the forearm connected to the elbow are different, and sizes of the thigh and the crus connected to the knee are different, therefore, the splint 100 has a better fit to the elbow and the connected upper arm and forearm, and to the knee and the connected thigh and crus, and the fixing and protection effects are better.

It should be noted that, in the embodiments of the present disclosure, the number of the plate comprising two sub-plates that are relatively rotatable is not limited, which can be determined according to actual needs. The more the plates comprising two sub-plates that are relatively rotatable are, the easier the bending angle of the splint 100 is maintained, and the fixing and protection effect of the bending portion is better. For example, in some examples, as illustrated in FIG. 1, each plate of the plurality of plates 110 comprises two sub-plates that are relatively rotatable. Moreover, the first plate 111 and the first plate 113 are oppositely disposed, and the second plate 112 and the second plate 114 are oppositely disposed. The first plate 111 is adjacent to the second plate 112, and the first plate 111 is adjacent to the second plate 114. The second plate 112 is adjacent to the first plate 111, and the second plate 112 is adjacent to the first plate 113. This arrangement makes the bending angle of the splint 100 easier to maintain, and the fixing and protection effect of the bending portion is better. For example, in some embodiments, only the first plate 111 and/or 113 may be provided with two sub-plates that are relatively rotatable, and the second plates 112 and 114 illustrated in FIG. 1 are replaced by two separate plates, so as to reduce the complexity of the splint 100, simplify the design, and reduce production costs.

In some embodiments of the present disclosure, for example, the splint 100 further comprises a pulling force detection circuit 130. The pulling force detection circuit 130 is coupled to the drawstring 121 and is configured to detect a pulling force applied to the drawstring 121. In a case where the number of the drawstrings 121 is plural, the pulling force detection circuits 130 may be provided in plurality, so as to respectively detect the pulling force of each of the drawstrings 121. For example, the pulling force detection circuit 130 may be disposed on any one of the plates, for example, disposed on the first plate 111. In order to cooperate with the pulling force detection circuit 130 to realize the pulling force detection function, the drawstring 121 may adopt a structure in which an insulating sheath wraps a core wire. The cross section of the core wire can be deformed under the pulling force, so that the resistance value of the core wire changes. The pulling force detection circuit 130 detects the resistance value of the core wire, and obtains a corresponding pulling force value by analyzing and calculating. For example, the specific relationship between the change value of the resistance and the deformation degree of the core wire, and the specific relationship between the deformation degree and the pulling force value can be obtained by theoretical calculation or test. For example, the material of the core wire may be a piezoelectric ceramic fiber composite material, a conductive rubber, or other suitable materials.

It should be noted that, in the embodiments of the present disclosure, the pulling force detection circuit 130 can be used to detect a resistance value, a capacitance value, or any other parameters, which may be determined according to the structure of the drawstring 121, and the embodiments of the present disclosure are not limit thereto. The pulling force detection circuit 130 can be implemented as a dedicated or general-purpose electronic hardware (or circuit), such as a conventional resistance detection circuit, a capacitance detection circuit, or the like, which is not limited in the embodiments of the present disclosure. For example, the pulling force detection circuit 130 may further comprise a calculation and processing circuit (for example, a processor) for calculating the corresponding pulling force value according to the detected resistance value, capacitance value, or the like. The specific configuration of the above electronic hardware is not limited, and may comprise an analog device, a digital chip, or other suitable devices.

In some embodiments of the present disclosure, for example, the splint 100 further comprises a display component 140. The display component 140 is disposed on one of the plurality of plates 110, for example, on the first plate 111. The display component 140 is in signal connection with the pulling force detection circuit 130 and is configured to display a pulling force detection result of the pulling force detection circuit 130. For example, the pulling force detection result comprises one or more of parameters such as a pulling force value, a recommended pulling force range, an indication of tightness degree, and so on. The display component 140 receives the pulling force detection result transmitted from the pulling force detection circuit 130 in a wire manner or a wireless manner, and displays in an appropriate manner.

For example, the display component 140 can be a display screen, a pointer instrument panel (such as a mechanical meter or a digital tube), or other suitable display devices, which is not limited in the embodiments of the present disclosure. The display screen is, for example, an organic light emitting diode (OLED) display screen, a liquid crystal display (LCD) screen, an electronic paper display screen, or the like. The size of the display component 140 is not limited and can be determined according to actual needs, for example, according to factors such as the size of the plate, the amount of display content, and so on.

By using the pulling force detection circuit 130 and the display component 140, the splint 100 can facilitate the user to monitor the tightness degree, help the user to adjust the appropriate tightness degree, and prevent the looseness of the splint 100, thereby improving the user's experience.

It should be noted that, in the embodiments of the present disclosure, the arrangement positions of the fastening drive device 122, the pulling force detection circuit 130, and the display component 140 are not limited, and may be respectively disposed on any suitable plate, and the three may be disposed on the same plate or different plates. For example, the pulling force detection circuit 130 and the display component 140 may be integrated as a whole, so as to reduce the number of components in the splint 100.

It should be noted that, in some embodiments of the present disclosure, the splint 100 may comprise more or less components, which may be determined according to actual needs, and the embodiments of the present disclosure are not limited thereto. For example, in some embodiments, the pulling force detection circuit 130 and the display component 140 may be omitted, and the splint 100 only comprises the plurality of plates 110 and the splint fastening device 120. For example, in other embodiments, the splint 100 may further comprise a reminder device (such as a buzzer) which is configured to send a reminder to the user, for example, an alarm sound, in a case where the splint is loose, that is, in a case where the pulling force value detected by the pulling force detection circuit 130 is less than a preset value. For example, in still other embodiments, the splint 100 can further comprise a power source, and the power source is configured to provide power to the pulling force detection circuit 130 and the display component 140, and in the case where the splint fastening device 120 comprises a motor, the motor can also be powered.

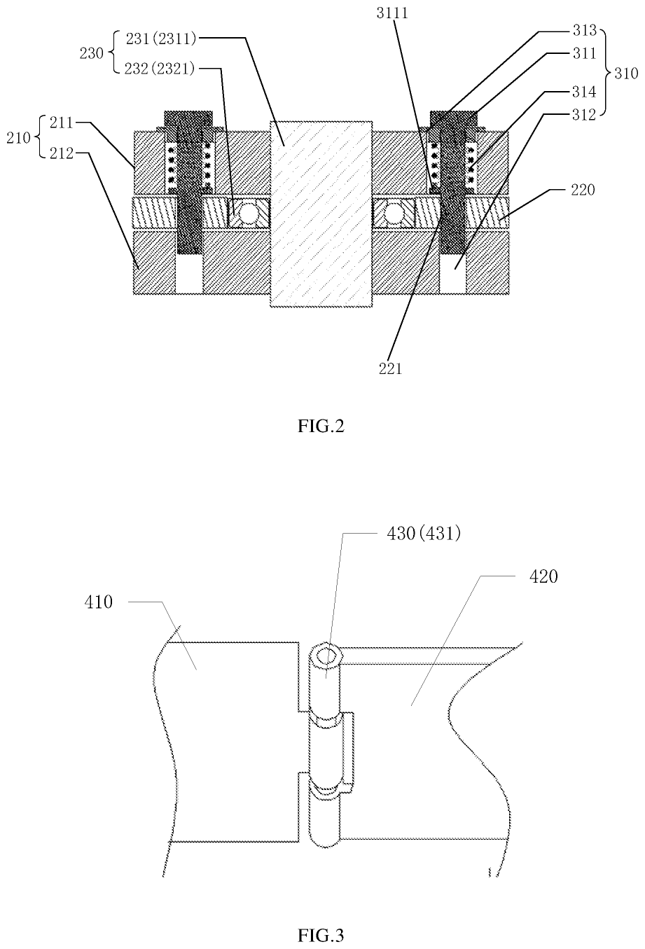

FIG. 2 is a sectional schematic diagram of a first rotating joint of the splint illustrated in FIG. 1. As illustrated in FIG. 2, the first rotating joint 230 comprises a fixing unit 231 and a rotating unit 232. The fixing unit 231 is connected to one of the first sub-plate 210 and the second sub-plate 220, and the rotating unit 232 is connected to the fixing unit 231 and the other one of the first sub-plate 210 and the second sub-plate 220.

For example, in some examples, the fixing unit 231 is a fixed shaft 2311, and the fixed shaft 2311 is fixed on one of the first sub-plate 210 and the second sub-plate 220, for example, fixed on the first sub-plate 210. The rotating unit 232 is a bearing 2321. An inner ring of the bearing 2321 is fixedly connected to the fixed shaft 2311, for example, connected integrally to a whole. An outer ring of the bearing 2321 is fixedly connected to the other one of the first sub-plate 210 and the second sub-plate 220, for example, connected integrally to a whole, for example, fixedly connected to the second sub-plate 220.

A portion, which is connected to the first rotating joint 230, of the first sub-plate 210 comprises a first connecting portion 211 and a second connecting portion 212 that are stacked, and a hollow structure is formed between the first connecting portion 211 and the second connecting portion 212. A portion, which is connected to the first rotating joint 230, of the second sub-plate 220 is located in the hollow structure. The first sub-plate 210 and the second sub-plate 220 are in surface contact with each other.

In a case where forces in opposite directions are respectively applied to the first sub-plate 210 and the second sub-plate 220, and the forces in opposite directions are in a plane in which a contact surface between the first sub-plate 210 and the second sub-plate 220 is located, the first sub-plate 210 and the second sub-plate 220 respectively drive the inner ring and the outer ring of the bearing 2321 to rotate relative to each other, thereby achieving relative rotation of the first sub-plate 210 and the second sub-plate 220.

For example, the splint 100 further comprises at least one positioning component 310. The positioning component 310 is disposed on the first sub-plate 210 for maintaining the bending angle of the splint 100, for example, the bending angle of the first sub-plate 210 and the second sub-plate 220.

For example, the positioning component 310 comprises a pin 311, a pin hole 312, a hollow bolt 313, and a spring 314. The pin hole 312 passes through the first connecting portion 211 and the second connecting portion 212. The pin 311 is in the pin hole 312 and is linearly movable in the pin hole 312. The pin 311 comprises a convex plate 3111. The hollow bolt 313 is disposed on an end of the pin hole 312, and the pin 311 is in the hollow bolt 313. One end of the spring 314 is connected to the hollow bolt 313, and the other end of the spring 314 is connected to the convex plate 3111. With the above configuration, the pin 311 can be connected to the first sub-plate 210 and keep attached to the surface of the first sub-plate 210 without being subjected to an external force. In a case where the pin 311 is subjected to an external force, the pin 311 can move linearly in a certain range to achieve the positioning function.

The portion, which is connected to the first rotating joint 230, of the second sub-plate 220 comprises a plurality of through holes 221. The plurality of through holes 221 are configured to allow the first sub-plate 210 and the second sub-plate 220 to be relatively fixed and respectively have a plurality of different bending angles in the case where the pin 311 is respectively inserted into the plurality of through holes 221. For example, in some examples, as illustrated in FIG. 1, four through holes 221 are non-uniformly distributed along a circular arc, so that the first sub-plate 210 and the second sub-plate 220 can have four different bending angles. Of course, the embodiments of the present disclosure are not limited thereto, and the through holes 221 may also be two, three, or any other number, which may be determined according to the number of required different bending angles. The distribution position and distribution manner of the plurality of through holes 221 are not limited, and the plurality of through holes 221 may be uniformly distributed or non-uniformly distributed, which may be determined according to actual needs.

For example, a length of the pin 311 is greater than a sum of a thickness of the first connecting portion 211 and a thickness of the hollow structure, or greater than a sum of a thickness of the second connecting portion 212 and a thickness of the hollow structure, so that the pin 311 can pass through the through hole 221, thereby fixing the relative position of the first connecting portion 211 and the second sub-plate 220, or fixing the relative position of the second connecting portion 212 and the second sub-plate 220, so as to fix the bending angle of the first sub-plate 210 and the second sub-plate 220.

It should be noted that, in the embodiments of the present disclosure, the number of the positioning components 310 is not limited, and may be, for example, any number such as one, two, three, etc., which may be determined according to actual needs. The greater the number of the positioning members 310 is, the stronger the connection between the first sub-plate 210 and the second sub-plate 220 is, and the easier the bending angle is to be maintained. For example, the number of the positioning components 310 may be less than or equal to the number of the through holes 221.

FIG. 3 is a partial enlarged diagram of a second rotating joint of the splint illustrated in FIG. 1. As illustrated in FIG. 3, the second rotating joint 430 may be a hinge structure 431. The third sub-plate 410 and the fourth sub-plate 420 are both connected to the hinge structure 431, and are configured to adopt the hinge structure 431 as a rotating axis to rotate with respect to the hinge structure 431. It should be noted that rotation manners of the second rotating joint 430 and the first rotating joint 230 are different, that is, the rotation manner of the third sub-plate 410 and the fourth sub-plate 420 and the rotation manner of the first sub-plate 210 and the second sub-plate 220 are different, so that the bending direction of each plate in the space is the same, and the splint 100 can be bent and restored as a whole.

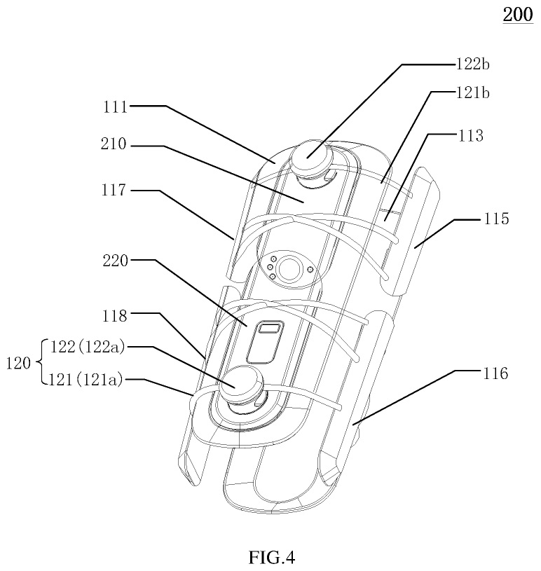

FIG. 4 is a stereoscopic schematic diagram of another splint provided by some embodiments of the present disclosure. As illustrated in FIG. 4, a splint 200 provided by this embodiment is substantially the same as the splint 100 as illustrated in FIG. 1 except for third plates 115-118. In this embodiment, the splint 200 comprises first plates 111 and 113, and third plates 115-118, and does not comprises the second plates 112 and 114 in the splint 100 illustrated in FIG. 1. The first plates 111 and 113 of the splint 200 are similar to the first plates 111 and 113 in the splint 100 illustrated in FIG. 1, and are not described here again.

The third plates 115-118 are four plates separated from each other. For example, the third plates 115 and 117 are connected to the first sub-plate 210 of the first plate 111 through the second drawstring 121b, and the third plates 116 and 118 are connected to the second sub-plate 220 of the first plate 111 through the first drawstring 121a. The bending and restoring of the splint 200 is achieved by the relative rotation of the first sub-plate 210 and the second sub-plate 220. Because the third plates 115-118 are separated from each other, the third plates 115-118 do not affect the bending and restoring of the splint 200. Compared with the splint 100 illustrated in FIG. 1, the splint 200 has a simple structure, which simplifies the design and reduces the production cost.

At least one embodiment of the present disclosure further provides an operation method of the splint according to any one of the embodiments of the present disclosure, and the splint provided by the embodiments of the present disclosure can be operated by using the operation method, the bending angle can be adjusted and the splint can be fixed, and the applications are wide. The operation method can enable the user to adjust quickly and easily, which reduces the workload of the user, improves the work efficiency, and improves the user's experience. The splint provided by at least some embodiments can also monitor tightness degrees.

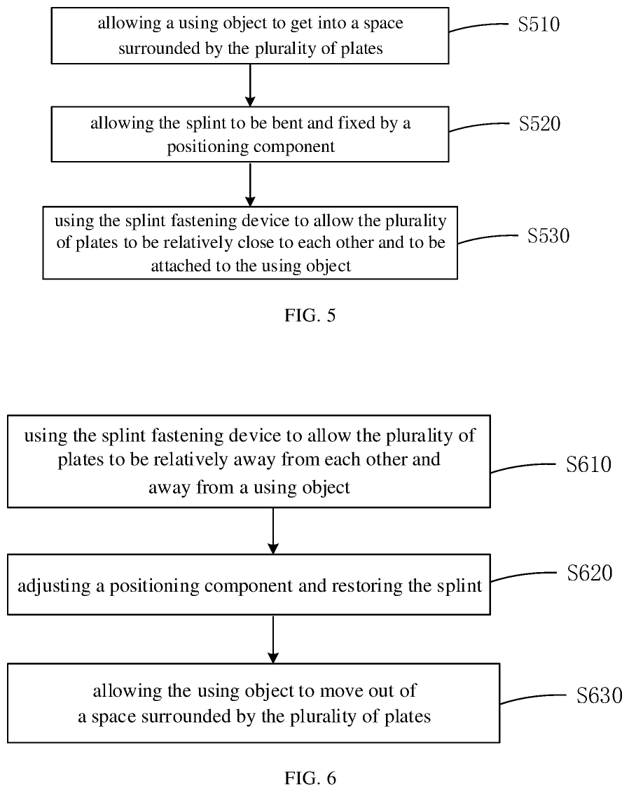

FIG. 5 is a flow diagram of an operation method of a splint provided by some embodiments of the present disclosure. For example, in some examples, as illustrated in FIG. 5, the operation method of the splint comprises following operations.

Step S510: allowing a using object to get into a space surrounded by the plurality of plates;

Step S520: allowing the splint 100/200 to be bent and fixed by a positioning component 310; and

Step S530: using the splint fastening device 120 to allow the plurality of plates 110 to be relatively close to each other and to be attached to the using object.

For example, the using object is a limb of the user, for example, an injured limb of the patient, such as a leg, an arm, or the like. For example, in a case where the splint 100/200 is bent, the bending angle of the splint 100/200 is substantially the same as the bending angle of the user's elbow, knee, or the like.

FIG. 6 is a flow diagram of another operation method of a splint provided by some embodiments of the present disclosure. For example, in some examples, as illustrated in FIG. 6, the operation method of the splint comprises following operations.

Step S610: using the splint fastening device 120 to allow the plurality of plates 110 to be relatively away from each other and away from an using object;

Step S620: adjusting a positioning component 310 and restoring the splint 100/200; and

Step S630: allowing the using object to move out of a space surrounded by the plurality of plates 110.

For details and technical effects of the above operation method, reference may be made to the above descriptions of the splint 100/200, and details are not described here again.

The following several statements should be noted.

(1) The accompanying drawings involve only the structure(s) in connection with the embodiment(s) of the present disclosure, and other structure(s) can be referred to common design(s).

(2) In case of no conflict, embodiments of the present disclosure and the features in the embodiments may be mutually combined to obtain new embodiments.

The above descriptions are only specific embodiments of the present disclosure, but the protection scope of the present disclosure is not limited thereto, the protection scope of the present disclosure should be determined by the protection scope of the claims.

What is claimed is:

1. A splint, comprising:
a plurality of plates; and
a splint fastening device connected to the plurality of plates and configured to allow the plurality of plates to be relatively close to each other or relatively away from each other,
wherein at least one plate of the plurality of plates comprises two sub-plates that are relatively rotatable, and the splint is capable of being bent or restored by relative rotation of the two sub-plates.

2. The splint according to claim 1, wherein the at least one plate comprises a first plate, and the first plate comprises a first sub-plate, a second sub-plate, and a first rotating joint,
the first sub-plate and the second sub-plate are both connected to the first rotating joint, and the first sub-plate and the second sub-plate are in surface contact with each other, and are relatively rotatable in a plane in which a contact surface between the first sub-plate and the second sub-plate is located.

3. The splint according to claim 2, wherein the first rotating joint comprises a fixing unit and a rotating unit,
the fixing unit is connected to one of the first sub-plate and the second sub-plate, and the rotating unit is connected to the fixing unit and another one of the first sub-plate and the second sub-plate.

4. The splint according to claim 3,
wherein the fixing unit comprises a fixing shaft, and the fixing shaft is fixed on one of the first sub-plate and the second sub-plate,
the rotating unit comprises a bearing, an inner ring of the bearing is fixedly connected to the fixing shaft, and an outer ring of the bearing is fixedly connected to another one of the first sub-plate and the second sub-plate.

5. The splint according to claim 4,
wherein a portion, which is connected to the first rotating joint, of the first sub-plate comprises a first connecting portion and a second connecting portion that are stacked, a hollow structure is between the first connecting portion and the second connecting portion, and
a portion, which is connected to the first rotating joint, of the second sub-plate is in the hollow structure.

6. The splint according to claim 5, further comprising at least one positioning component, wherein the positioning component is on the first sub-plate.

7. The splint according to claim 6, wherein the positioning component comprises a pin and a pin hole,
the pin hole extends through the first connecting portion and the second connecting portion, and the pin is in the pin hole and is linearly movable in the pin hole, and
a length of the pin is greater than a sum of a thickness of the first connecting portion and a thickness of the hollow structure, or greater than a sum of a thickness of the second connecting portion and a thickness of the hollow structure.

8. The splint according to claim 7, wherein the positioning component further comprises a hollow bolt and a spring, and the pin comprises a convex plate,
the hollow bolt is on one end of the pin hole, and the pin is in the hollow bolt, one end of the spring is connected to the hollow bolt, and another end of the spring is connected to the convex plate.

9. The splint according to claim 7, wherein the portion, which is connected to the first rotating joint, of the second sub-plate comprises a plurality of through holes, and
the plurality of through holes are configured to allow the first sub-plate and the second sub-plate to be relatively fixed and respectively have a plurality of different bending angles in a case where the pin is respectively inserted into the plurality of through holes.

10. The splint according to claim 1, wherein the at least one plate comprises a second plate,
the second plate comprises a third sub-plate, a fourth sub-plate, and a second rotating joint, and
the third sub-plate and the fourth sub-plate are both connected to the second rotating joint, and are configured to adopt the second rotating joint as a rotating axis to rotate with respect to the second rotating joint.

11. The splint according to claim 10, wherein the second rotating joint comprises a hinge structure.

12. The splint according to claim 1, wherein the at least one plate comprises a plurality of first plates and a plurality of second plates,
each of the plurality of first plates comprises a first sub-plate, a second sub-plate, and a first rotating joint, the first sub-plate and the second sub-plate are both connected to the first rotating joint, and the first sub-plate and the second sub-plate are in surface contact with each other and are relatively rotatable in a plane in which a contact surface between the first sub-plate and the second sub-plate is located,
each of the plurality of second plates comprises a third sub-plate, a fourth sub-plate, and a second rotating joint, and the third sub-plate and the fourth sub-plate are both connected to the second rotating joint, and are configured to adopt the second rotating joint as a rotating axis to rotate with respect to the second rotating joint,
the plurality of first plates are oppositely disposed, the plurality of second plates are oppositely disposed, and the first plates are adjacent to the second plates.

13. The splint according to claim 1, wherein the splint fastening device comprises:
at least one drawstring movably connected to the plurality of plates; and
at least one fastening drive device on one or more of the plurality of plates, connected to the at least one drawstring and configured to be controllable to drive the at least one drawstring, so that the drawstring is capable of being tightened or relaxed under driving function of the fastening drive device to drive the plurality of plates to be relatively close to each other or relatively away from each other.

14. The splint according to claim 13, wherein the at least one drawstring comprises a plurality of drawstrings, and the at least one fastening drive device comprises a plurality of fastening drive devices, the plurality of drawstrings are connected to the plurality of fastening drive devices in one-to-one correspondence, and
the plurality of drawstrings are movably connected to plates of different portions of the splint that are capable of being bent, respectively.

15. The splint according to claim 13, further comprising a pulling force detection circuit,
wherein the pulling force detection circuit is coupled to the drawstring and is configured to detect a pulling force applied to the drawstring.

16. The splint according to claim 15, further comprising a display component,
wherein the display component is in signal connection with the pulling force detection circuit, and is configured to display a pulling force detection result of the pulling force detection circuit.

17. The splint according to claim 8, wherein the portion, which is connected to the first rotating joint, of the second sub-plate comprises a plurality of through holes, and the plurality of through holes are configured to allow the first sub-plate and the second sub-plate to be relatively fixed and respectively have a plurality of different bending angles in a case where the pin is respectively inserted into the plurality of through holes;
the at least one plate comprises a second plate, the second plate comprises a third sub-plate, a fourth sub-plate, and a second rotating joint, and the third sub-plate and the fourth sub-plate are both connected to the second rotating joint, and are configured to adopt the second rotating joint as a rotating axis to rotate with respect to the second rotating joint, and the second rotating joint comprises a hinge structure;
the at least one plate comprises a plurality of first plates and a plurality of second plates, the plurality of first plates are oppositely disposed, the plurality of second plates are oppositely disposed, and the first plates are adjacent to the second plates;
the splint fastening device comprises: at least one drawstring movably connected to the plurality of plates; and at least one fastening drive device on one or more of the plurality of plates, connected to the at least one drawstring and configured to be controllable to drive the at least one drawstring, so that the drawstring is capable of being tightened or relaxed under driving function of the fastening drive device to drive the plurality of plates to be relatively close to each other or relatively away from each other;
the at least one drawstring comprises a plurality of drawstrings, and the at least one fastening drive device comprises a plurality of fastening drive devices, the plurality of drawstrings are connected to the plurality of fastening drive devices in one-to-one correspondence, and the plurality of drawstrings are movably connected to plates of different portions of the splint that are capable of being bent, respectively;
the splint further comprises a pulling force detection circuit, and the pulling force detection circuit is coupled to the drawstring and is configured to detect a pulling force applied to the drawstring; and
the splint further comprises a display component, and the display component is in signal connection with the pulling force detection circuit, and is configured to display a pulling force detection result of the pulling force detection circuit.

18. The splint according to claim 8, wherein the portion, which is connected to the first rotating joint, of the second sub-plate comprises a plurality of through holes, and the plurality of through holes are configured to allow the first sub-plate and the second sub-plate to be relatively fixed and respectively have a plurality of different bending angles in a case where the pin is respectively inserted into the plurality of through holes;
the splint fastening device comprises: at least one drawstring movably connected to the plurality of plates; and at least one fastening drive device on one or more of the plurality of plates, connected to the at least one drawstring and configured to be controllable to drive the at least one drawstring, so that the drawstring is capable of being tightened or relaxed under driving function of the fastening drive device to drive the plurality of plates to be relatively close to each other or relatively away from each other;
the at least one drawstring comprises a plurality of drawstrings, and the at least one fastening drive device comprises a plurality of fastening drive devices, the plurality of drawstrings are connected to the plurality of fastening drive devices in one-to-one correspondence, and the plurality of drawstrings are movably connected to plates of different portions of the splint that are capable of being bent, respectively;
the splint further comprises a pulling force detection circuit, and the pulling force detection circuit is coupled to the drawstring and is configured to detect a pulling force applied to the drawstring; and
the splint further comprises a display component, and the display component is in signal connection with the pulling force detection circuit, and is configured to display a pulling force detection result of the pulling force detection circuit.

19. An operation method of the splint according to claim 1, comprising:
allowing a using object to get into a space surrounded by the plurality of plates;
allowing the splint to be bent and fixed by a positioning component; and
using the splint fastening device to allow the plurality of plates to be relatively close to each other and to be attached to the using object.

20. An operation method of the splint according to claim 1, comprising:
using the splint fastening device to allow the plurality of plates to be relatively away from each other and away from a using object;
adjusting a positioning component and restoring the splint; and
allowing the using object to move out of a space surrounded by the plurality of plates.
